(19) United States
(12) Patent Application Publication
(10) Pub. No.: US 2008/0033260 A1
(43) Pub. Date: Feb. 7, 2008

(54) Cardiac Biosensor Devices and Methods

(75) Inventors: Norman F. Sheppard, New Ipswich, NH (US); John T. Santini, North Chelmsford, MA (US)

Correspondence Address: SUTHERLAND ASBILL & BRENNAN LLP, 999 PEACHTREE STREET, N.E., ATLANTA, GA, 30309, US

(73) Assignee: MICROCHIPS, INC., Bedford, MA (US)

(21) Appl. No.: 11/833,011

(22) Filed: Aug. 2, 2007

Related U.S. Application Data
(60) Provisional application No. 60/821,351, filed on Aug. 3, 2006.

(51) Int. Cl. A61B 5/02

(52) U.S. Cl. 600/301

(57) ABSTRACT

Implantable medical devices for cardiac care are provided that include a housing having a power source and control electronics; at least one lead extending from the housing and having one or more discrete reservoirs therein, each reservoir having an opening to an outer surface of the lead; one or more sensors, which monitor or detects an analyte, biomarker, or physical parameter that is associated with cardiac health, located in the reservoirs and in operable communication with said control electronics; and at least one selectively disintegratable reservoir cap sealing each of the reservoir openings, wherein the reservoir cap is operably connected to the power source and control electronics to disintegrate the reservoir cap and expose the sensors in vivo. The sensor may detect an analyte or biomarker selected from potassium ion, sodium ion, lithium ion, magnesium ion, ammonium ion, ionized calcium, lactate, oxygen, carbon dioxide, and creatinine, urea, BUN, and bilirubin.

### CROSS-REFERENCE TO RELATED APPLICATIONS

This application claims benefit of U.S. Provisional Application No. 60/821,351, filed Aug. 3, 2006. The application is incorporated herein by reference.

### BACKGROUND OF THE INVENTION

This invention relates generally to medical devices for sensing, and more particularly to medical devices for physiological sensing in vivo for cardiac care. Prior art medical devices are disclosed in U.S. Pat. No. 6,551,838 to Santini, Jr. et al. and U.S. Patent Application Nos. 2006/0100608 to Uhland et al., 2006/0057737 to Santini, Jr. et al., and 2004/0106953 to Yomtov et al.

Heart disease is a leading cause of mortality and morbidity worldwide, and its manifestations include heart failure, acute coronary syndromes and arrhythmias.

Heart failure is a progressive disease with either structural or functional etiology that results in ventricular remodeling, increased stress on the ventricular wall and decreased pumping efficiency. Diagnosed through physical examination, echocardiography and laboratory testing, a heart failure patient typically is treated with a combination of medications and lifestyle modifications. Selected heart failure patients may be candidates for biventricular pacing and cardiac resynchronization devices, as well as for implantable hemodynamic monitoring devices.

Heart failure patients may be treated with a combination of ACE inhibitors, angiotensin receptor blockers, aldosterone blockers, beta blockers and digoxin. These treatments have side effects including increased risk of hyperkalemia, which can lead to arrhythmias and impaired renal function. Even with medications, a heart failure patient may go into acute decompensation, which is a life-threatening complication usually requiring hospitalization.

Measurements of electrolytes, metabolites, blood gases, and cardiac biomarkers in the patient can provide diagnostic and risk stratification information to cardiologists. Measurement of electrolytes and other molecules such as potassium, sodium, calcium, and hydrogen can help in monitoring the health status and drug therapy effects in heart failure patients. For example, monitoring potassium levels can indicate whether the patient has the correct mix and dosing of medications. Monitoring metabolites such as glucose, urea, and creatinine can assist in managing a patient who is a diabetic or who has renal dysfunction. Monitoring the patient's blood gases can provide an indication of oxygenation levels, for example.

Conventional point-in-time assessments, however, are limited in that they cannot offer monitoring or predictive information about a patient's worsening condition that could lead to a change in medications or patient management. It therefore would be desirable to have implanted biosensors capable of measuring a heart failure patient's electrolytes, metabolites and blood gases, preferably continually, frequently, and/or over an extended period.

B-type, or brain, natriuretic peptide (BNP) and pro-BNP levels are correlated with left ventricular overload and are predictive of heart failure in post-myocardial infarction patients (Bettencourt, et al., Clin Cardiol., 23:921-27 (2000)). Measuring BNP and other markers, such as norpinephrine, can lead to improvements in clinical care for patients at risk of acute decompensation and hospitalization resulting from left ventricular dysfunction (Bozkurt & Mann, Circulation 107:1231 (2003)). It therefore would also be desirable to have implanted biosensors for the detection of cardiac biomarkers implicated in heart failure.

Acute coronary syndromes and myocardial ischemia refer to a cascade of events whereby plaque within the coronary arteries is disrupted, causing an acute thrombotic release that blocks blood flow within the artery and starves the myocardium of oxygen-rich blood. Poor oxygenation of the heart muscle causes myocardial necrosis that often leads to impaired cardiac function. Acute coronary syndromes (ACS) patients have myocardial infarction or the unstable angina that is often an indication of myocardial ischemia. Acute coronary syndromes is typically the result of a cascade of events beginning with atherosclerosis and leading to myocardial ischemia and eventually to acute episodes based on rupture of lipid-rich pools of atherosclerotic plaque.

Suspected ACS patients have a physical exam and an EKG to look for ST-segment elevation. Testing is performed to took for biomarkers of myocardial necrosis, which include cardiac troponin (I and T), creatine kinase (CK), myoglobin, and lactate dehydrogenase. These biomarkers are often used to determine whether a plaque rupture has already occurred, although circulating cardiac troponin levels have predictive value for cardiac events. (Hamm, et al., N Engl J Med. 327:146-50 (1992); Hamm et al., Circulation 102:118-22 (2000); and Heidenreich, et al., J Am Coll Cardiol. 38:478-85 (2001)).

Current treatment of ACS patients depends on the presence of ST segment elevation, but often includes pharmacological agents such anti-thrombotics, low molecular weight heparin, glycoprotein IIa/IIIb inhibitors and ACE inhibitors. Percutaneous interventions such as balloon angioplasty and stent placement may be performed to reopen and maintain blood flow within the vasculature. Surgical grafting of the coronary arteries may also be used to bypass blockages that are not suitable for percutaneous treatments.

Lactate levels can be an early indicator of myocardial ischemia and rising lactate levels could prompt clinical intervention in advance of a myocardial infarction. Inflammation markers, such as CRP and IL-6 among others, may provide an early warning of the factors that lead to the rupture of vulnerable plaque and to myocardial infarction. It therefore would be desirable to have an implanted sensor for the detection of electrolytes related to acute coronary syndromes and myocardial infarction. It also would be desirable to have an implant capable of detecting cardiac biomarkers related to ACS.

The electrical and conduction systems of the heart regulate cardiac contractions and the heart's pumping action. Arrhythmias are abnormal rhythms that disturb the timing and synchronization of the heart's pumping and can lead to a variety of conditions, including life threatening cardiac arrest which occurs in approximately 400,000 people annually in the United States. Arrhythmias may be detected by electrocardiography, by holter tests, by stress tests, and by electrophysiology studies. Myocardial infarction and congestive heart failure patients, as well as patient with coronary artery disease, are at elevated risk of sudden cardiac arrest.

Biochemical imbalances, especially electrolyte imbalances, can cause arrhythmias, and cardiac resuscitation therapy can be ineffective in restoring normal sinus rhythm absent electrolyte re-balancing (Alfonzo, et al., Resuscitation 70:10-25 (2006)).

Treatments for arrhythmias include anti-arrhythmic drugs such as beta blockers, amiodarone, qunidine, procainamide, disopyramide and bretylium. If the arrhythmia is atrial fibrillation, the patient may also receive antithrombotic or anticoagulant therapy to prevent blood clots leading to stroke. Arrhythmia patients often have implanted pacemakers and cardiac defibrillators that detect abnormal heart rhythms and shock the heart back into normal sinus rhythm. Patients may undergo cardiac ablation procedures to remap the electrical conduction pathways of the heart.

Pharmacologic treatments for anti-arrhythmia typically have side effects. For example, beta blockers have been linked to an increased risk of type-2 diabetes mellitus (Stump, et al., Mayo Clin Proc. 81(6):796-806 (2006). Diabetes patients on beta blocker therapy are encouraged to monitor their glucose levels more closely to avoid hypoglycemia (Cleland, Medical Clinics of North America, 87(2): 339-65 (2003)). As another example, amiodarone has been linked to thyroid disease and to liver and pulmonary toxicity. It therefore would be highly desirable to have an implantable sensor that can assess liver function and to otherwise monitor the effect of pharmacologic treatments.

Earlier detection of elevated or depressed potassium levels could indicate a higher risk of arrhythmia. It therefore would be desirable to have implanted biosensors capable of measuring electrolyte levels to provide additional clinical information for the management of arrhythinia patients. It would also be desirable to use implanted biosensors for detection of metabolites such as glucose in patients at risk of developing diabetes from anti-arrhythmia medications. It would be desirable to monitor additional metabolites and blood gases that may be affected by anti-arrhythmia medications. It would be useful to have implanted biosensors to measure cardiac biomarkers that are implicated in atherosclerotic diseases, inflammation, vulnerable plaque and myocardial ischemia. Sensing these analytes could help detect clinical precursors to myocardial infarction and allow for earlier and more effective interventions. Accordingly, it would be desirable to have biosensors for measuring relevant electrolytes, metabolites, blood gases and biomarkers in coronary artery disease, peripheral artery disease, peripheral venous disease and neurovascular disease.

In addition, it would be desirable to provide improved sensing devices, particularly implantable medical devices containing one or more biosensors, which can be operated for an extended period of time and which may include a variety of sensor types useful in cardiac therapies and cardiac health management.

### SUMMARY OF THE INVENTION

Improved implantable devices and sensing methods are provided. In one aspect, the implantable medical device comprises: a housing which includes a power source and control electronics; at least one lead extending from the housing and having one or more discrete reservoirs therein, each reservoir having at least one opening to an outer surface of the lead; one or more sensors, which monitor or detects in vivo an analyte, biomarker, or physical parameter that is associated with cardiac health, located in the one or more reservoirs and in operable communication with said control electronics; and at least one selectively disintegratable reservoir cap sealing each of said at least one opening of said one or more reservoirs, wherein the at least one reservoir cap is operably connected to the power source and control electronics to selectively disintegrate the reservoir cap and expose the one or more sensors in vivo. The one or more reservoirs may be located on a tip portion of the at least one lead. In one embodiment, the control electronics comprise a microprocessor or state machine.

In certain embodiments, the one or more sensors comprises a biosensor for the monitoring or detecting of an analyte or a biomarker selected from the group consisting of potassium ion, sodium ion, lithium ion, magnesium ion, ammonium ion, ionized calcium, lactate, oxygen, carbon dioxide, creatinine, urea, BUN, bilirubin, alkaline phosphatase, aspartate aminotransferase, alanine aminotransferase, lactic dehydrogenase, gamma glutamyl transpeptidase, heparin, warfarin, ischemia modified albumin (IMA), myeloperoxidase, matrix metalloproteinase (MMP), pH, and placental growth factor. In another embodiment, the one or more sensors or sensing components measures an ECG, an EKG, or another intrinsic electrical signal.

In one embodiment, the at least one lead further includes a secondary sensor located on an external surface of the at least one lead and not in the one or more reservoirs.

In a certain embodiment, the device may further include at least one secondary lead without a sensor. In one embodiment, the at least one lead and/or the secondary lead may further include an electrode for cardiac pacing, defibrillation, or neurostimulation. In another embodiment, the at least one lead and/or the secondary lead may further include one or more secondary reservoirs containing at least one drug for controlled release in vivo. The release of the at least one drug from the one or more secondary reservoirs may be passively or actively controlled.

In one embodiment, the device may further include a transmitter for communicating an electrical signal from the one or more sensors to a remote receiver. In one example, the remote receiver may be operably coupled to a controller for controlling delivery of a drug. In another example, the remote receiver may be operably coupled to a controller for controlling cardiac pacing, defibrillation, or neurostimulation.

In another aspect, a method is provided for monitoring a patient in need of cardiac care. In one embodiment, the method includes the steps of (i) implanting into a patient a medical device which comprises a housing which includes a power source and control electronics; at least one lead extending from the housing and having one or more discrete reservoirs therein, each reservoir having at least one opening to an outer surface of the lead; one or more sensors, which monitor or detects in vivo an analyte, biomarker, or physical parameter that is associated with cardiac health, located in the one or more reservoirs and in operable communication with said control electronics; and at least one selectively disintegratable reservoir cap sealing each of said at least one opening of said one or more reservoirs, wherein the at least one reservoir cap is operably connected to the power source and control electronics to selectively disintegrate the reservoir cap; (ii) disintegrating the at least one reservoir cap and exposing the one or more sensors in vivo; and (iii) using the one or more sensors following disintegration of the reservoir cap to monitor or detect an analyte, biomarker, or physical parameter associated with the health of the patient's heart.

In one embodiment, the analyte or biomarker is selected from the group consisting of potassium ion, sodium ion, lithium ion, magnesium ion, ammonium ion, ionized calcium, lactate, oxygen, carbon dioxide, creatinine, urea, BUN, bilirubin, alkaline phosphatase, aspartate aminotransferase, alanine aminotransferase, lactic dehydrogenase, gamma glutamyl transpeptidase, heparin, warfarin, ischemia modified albumin (IMA), myeloperoxidase, matrix metalloproteinase (MMP), pH, and placental growth factor.

In one embodiment, the physical parameter comprises a fluid flow rate, pressure, or viscosity. In another embodiment, the physical parameter comprises an ECG, EKG, or another intrinsic electrical signal.

In a certain embodiment, the medical device further includes an electrode for cardiac pacing, defibrillation, or neurostimulation. In another embodiment, the medical device further comprises a drug for controlled release in vivo. In one particular embodiment, the medical device comprises a plurality of discrete microreservoirs and a plurality of corresponding discrete reservoir caps which comprise a metal film and are mechanically and electrically connected to a pair of electrical leads. The electrical leads may be connected to the power source, which can deliver an electrical current through the reservoir cap to disintegrate it.

### BRIEF DESCRIPTION OF THE DRAWINGS

FIG. 1 is a schematic illustration of an implantable medical device according to one embodiment.

FIG. 2 is a cross-sectional view of the tip portion of a lead of an implantable medical device according to one embodiment.

FIG. 3 is a cross-sectional view of the tip portion of a lead of an implantable medical device according to one embodiment.

FIG. 4 is a cross-sectional view of the tip portion of a lead of an implantable medical device according to one embodiment.

FIG. 5 is a cross-sectional view of the tip portion of a lead of an implantable medical device according to one embodiment.

FIG. 6 is a cross-sectional view of the tip portion of a lead of an implantable medical device according to one embodiment.

FIG. 7 is a schematic illustration of an implantable medical device according to one embodiment.

FIG. 8 is a schematic illustration of an implantable medical device according to one embodiment.

### DETAILED DESCRIPTION OF THE INVENTION

Implantable reservoir-based sensor devices have been developed for use in cardiac care applications. Cardiac care, as used herein, refers generally to the monitoring and optional treatment of cardiovascular health, whether related to existing cardiovascular medical conditions or preventing of cardiovascular medical conditions. In particular, cardiac care refers to the diagnosis, treatment, or management of a cardiac disease, disorder, chronic condition, or failure. Thus, the reservoir-based sensor devices embodied herein include sensors or sensing components capable of monitoring or detecting in vivo analytes, biomarkers, or other physical parameters which may be indicative of cardiac disease, disorder, chronic condition, or failure.

In one aspect, the reservoir based devices provide chemical sensing capability with conventional cardiac implant devices, such as simple pacemakers, cardiac resynchronization therapy (CRT) pacemakers, defibrillators, implantable cardioverter defibrillators (ICDs), left ventricular assist devices (LVADs), heart monitors, hemodynamic monitors, Rheos™ (CVRx Inc.) hypertension treatment devices, percutaneous transvenous mitral annuloplasty (PTMA) devices, Swan-Ganz catheters, and other cardiac devices. The sensors can communicate with or be integrated into the existing cardiac implant devices. The chemical sensor functionality optionally can be coupled with drug delivery, which also may be multi-reservoir-based. The drug delivery feature may be a separate unit or an integral component of the cardiac implant device, and in either approach the drug may be released in vivo based on sensor output. The sensors may be wirelessly connected or hardwired to the cardiac device and/or to optional the drug delivery device or component.

In general, the implantable medical devices comprise at least one sensor device. The sensor device may include one or more reservoir devices. A typical reservoir device includes a body portion (i.e., a substrate), one or more reservoirs, one or more sensors stored in the reservoirs, and means for selectively opening the reservoir caps to expose the one or more sensors. By sealing the sensors in reservoirs, the sensors can be protected from the environment while on the shelf, and then can be controllably/selectively exposed to fluids when needed (e.g., following implantation in a patient). The reservoir structure also protects sensitive sensor components (e.g. enzymes) from the hostile in vivo environment until needed, thereby permitting the use of sensor chemistries that otherwise would be impractical or useless, for example due to their limited stability or shelf-life (if not protected in sealed reservoirs).

These reservoir-based sensors may be made into a stand alone, complete implantable device. For instance, an array of reservoirs containing biosensors could be provided in a substrate that is packaged in a housing with power and control electronics and wireless communication electronics. Alternatively, the reservoir-based sensor may be incorporated into, built into or added onto, an existing implant device or another type of implant device, such as a conventional pacemaker or hemodynamic monitor (e.g., CHRONICLE™ (Medtronic Inc.)). See, e.g., U.S. Pat. No. 5,535,752 and U.S. Pat. No. 5,564,434.

As used herein, the terms “comprise,” “comprising,” “include,” and “including” are intended to be open, non-limiting terms, unless the contrary is expressly indicated.

### The Device

For simplicity, only one or two reservoirs are shown in some Figures. However, it is understood that a reservoir array component or device may contain many more reservoirs. It also is understood that the number, geometry, and placement of each reservoir, reservoir cap, electrodes, or electrical traces may be modified for a particular application. It is envisioned that various reservoir activation means (active, passive, mechanical rupture, electrothermal ablation, etc.) can be used and combined in different device designs.

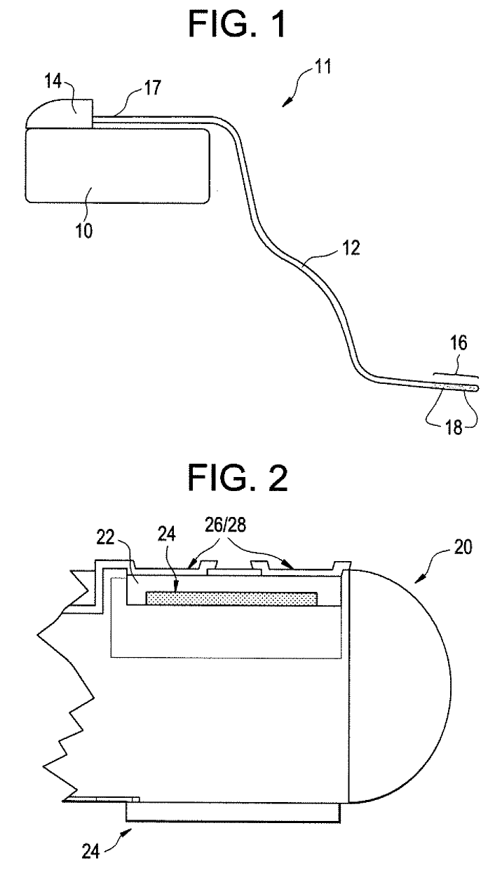

In one embodiment, shown in FIG. 1, the implant device 11 includes a disk- or puck-shaped housing 10, which contains the electronics and a power source (e.g., battery), and one or more flexible leads 12 extending from the housing via a hermetic feedthrough 14. A tip portion 16 of the lead 12 includes reservoirs 18 which contain sensors. The sensor may be a chemical sensor, a mechanical sensor, an electrical sensor, or any other sensor suitable for use in the monitoring of cardiovascular health.

Leads, as used herein, are elongated, flexible, tube-like structures that extend from a larger, substantially fixed and rigid device portion and connect to a target biological tissue, which typically is remote from the larger device portion and would otherwise generally be difficult to access directly with the device. Various implantable leads are known in the art and may be used in embodiments herein. Generally, the lead 12 has a distal end 16 and a proximal end 17. The distal end 16, which is synonymous with the term tip portion, generally is the portion of the lead which is furthest from the implant device and is adapted to physically and/or electrically contact body tissue at a desired location. The proximal end 17 generally is portion of the lead which is connected to the implant device. In particular embodiments, the lead may be used to electrically connect the implant device to a desired body tissue location.

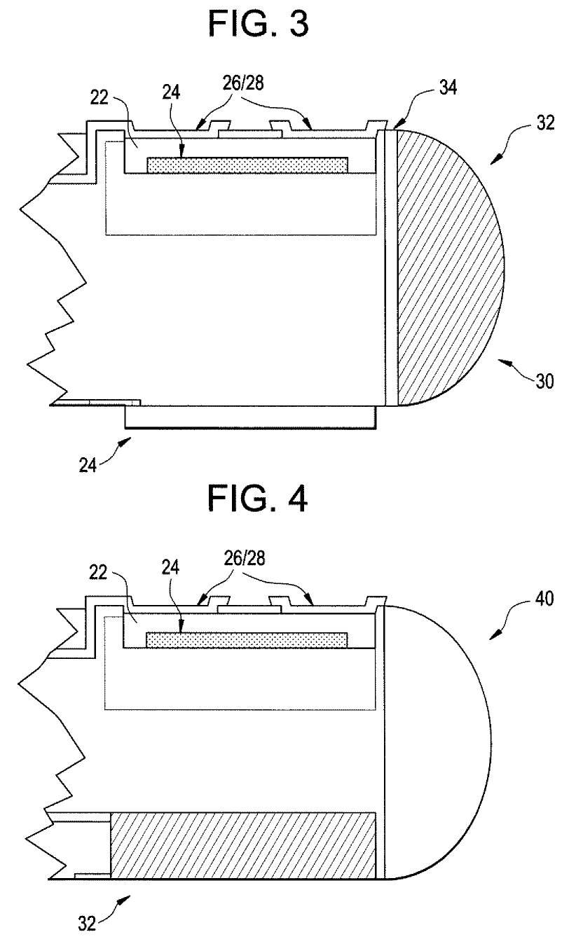

FIGS. 2 and 3 show cross-sectional views of two other embodiments of a tip portion 20 of a lead with a reservoir 22. Reservoir 22 contains a sensor 24 and has two openings 26 sealed by reservoir caps 28. The tip portion 20 also may include additional sensors or electrodes 24 that are located only partially in the reservoir or that are not located in the reservoir at all.

It is noted that only a portion of (e.g., a component of) the sensor need be located in the reservoir. For example, a reference electrode may be located on the substrate (body portion) nearby and outside of a reservoir that contains a working electrode, where the two electrodes together form a single sensor.

In use, the tip portion may be placed in the subcutaneous space, the intraperitoneal space, or the blood stream. In one embodiment, the tip portion is placed in a blood vessel near the heart or directly in one of the chambers of the heart-much like a conventional pacemaker lead. For example, the device may be designed solely as a cardiac sensing device, with each of the sensors providing continuous or discrete/limited sensing of a parameter/analyte of interest. In another embodiment, the cardiac implant device is adapted to pace or defibrillate, in addition to sense. Such a device would be particularly useful if the sensors could aid the physician (or the device) to better determine the cause of a cardiac problem, or if the sensor information could help determine the best course of therapy, with the help being delivered more quickly or more accurately than with conventional cardiac devices without implanted sensor capability. In one example, illustrated in FIGS. 3 and 4, the tip portion 30 and 40, respectively, of a lead includes an electrode 32 for electrical stimulation. The electrode 32 may be located at the end of the lead, as illustrated in FIG. 3, or on a side surface of the lead, as illustrated in FIG. 4. The tip portion 30 of the lead may further include an insulator 34.

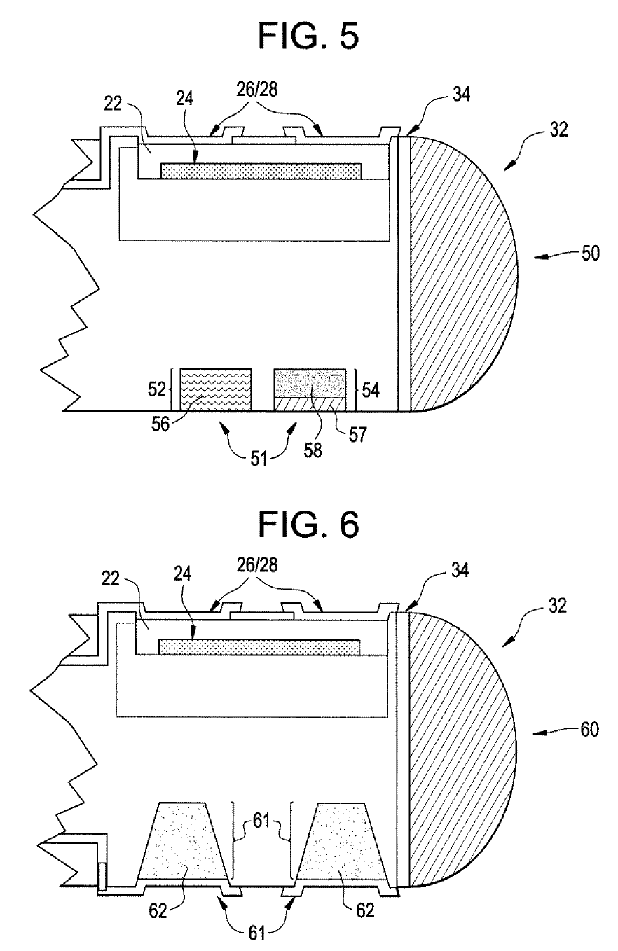

FIGS. 5 and 6 illustrate other embodiments of the tip portion of a lead of the implant device. In these figures, the tip portion includes an array of discrete secondary reservoirs containing one or more drugs for controlled release in vivo. FIG. 5 shows a cross-sectional view of tip portion 50 which includes secondary reservoirs 52 and 54 which provide passive controlled release of drug (in addition to the reservoir-based sensors and stimulation electrodes described in FIGS. 2 to 4). Reservoir 52 contains a first drug formulation 56, which may be a drug homogeneously dispersed in a matrix material (e.g., hydrophobic excipient, biodegradable polymer, etc.) from which the drug can be released upon diffusion through or dissolution/degradation of the matrix material. Reservoir 54 contains a second drug formulation 58, which is covered by a passive reservoir cap 57. Release of drug formulation 58 is initiated following in vivo dissolution/degradation of reservoir cap 57.

FIG. 6 shows a cross-sectional view of tip portion 60 which includes secondary reservoirs 61 which provide active controlled release of drug (in addition to the reservoir-based sensors and stimulation electrodes described in FIGS. 2 to 4). Reservoirs 61 contain drug formulation 62 and have openings covered by active reservoir caps 64. For example, reservoir caps 64 may be disintegrated by electrothermal ablation to initiate release of the drug 62 at a time indicated by sensor output. Drug release may be coordinated with delivery of electrical stimulation through the stimulation electrode 32.

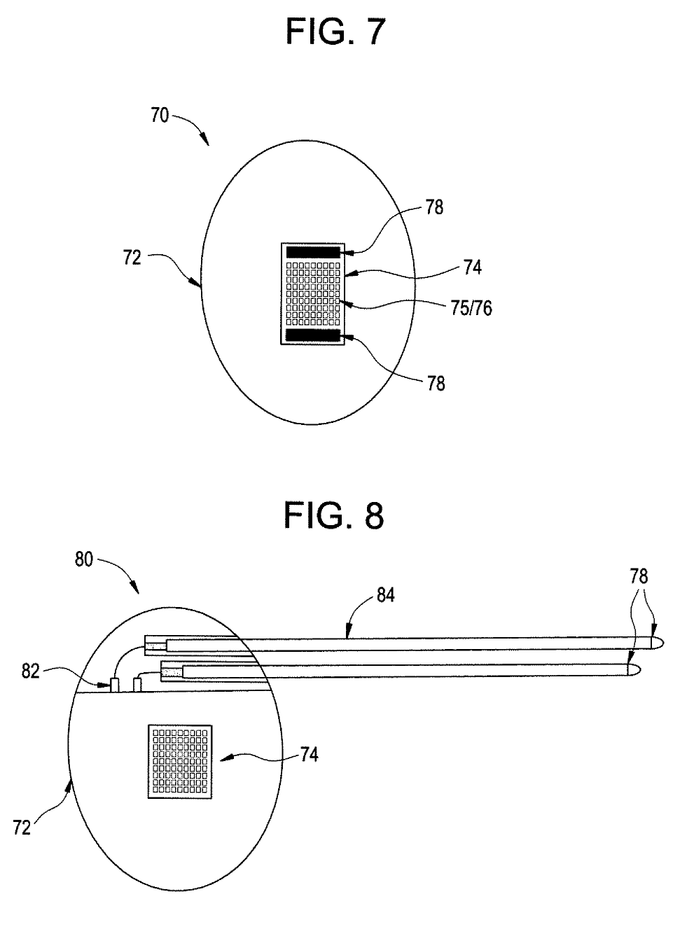

In another embodiment, the reservoirs and chemical sensors can be built into a planar or disc-shaped housing (and not placed at the tip of a lead). See FIGS. 7 and 8, for example. In one embodiment, the implantable device 70 comprises a housing 72 comprising a sensor module 74. The sensor module 74 may include a plurality of reservoirs 75 containing sensors 76. The device may optionally include ECG monitoring electrodes 78 integrated within the housing itself. This type of device design could be used for sensors that will monitor patient parameters in the subcutaneous space. Alternatively, the device 80 may comprise a plastic header with a hermetic feedthrough 82 through which one or more leads 84 extend. The leads 84 may optionally may include sensors (not shown) and/or ECG monitoring electrodes 78.

In one particular application illustrating the advantageous uses of the present devices, the sensor could be used to alert a physician that an arrhythmia patient's potassium levels are unbalanced (too high or too low). Standard resuscitation techniques (i.e. defibrillation) will not work unless the patient's potassium levels are in balance. Thus, the sensor could immediately notify the patient or physician and/or adjust pacing or defibrillation protocols automatically based on the currently sensed potassium levels in the patient.

Any of the foregoing sensor devices could be operably coupled to a drug delivery device, either multi-reservoir based or not (e.g., pump-based devices delivering fluidized drug from a single reservoir), to administer one or more drug therapies based on sensor output.

It is noted that the implant device (such as the one shown in FIG. 1) could be modified to include two or more separate leads, wherein the separate leads each provide a different (or possibly the same) functionality. For example, one lead could have a tip portion with reservoir-based sensors and another lead could have a tip portion with electrodes for electrical stimulation. As another example, one lead could sense and another could provide local, controlled drug delivery.

The sensor device may be packaged and implanted separately from the pacemaker/defibrillator device. However, the two device units preferably would be in communication with one another while in the body. Such a device system may be particularly advantageous because each unit may have different optimal sites for implantation and/or each unit may have a different working life (such that different explantation schedules are needed). The two units could communicate using any of a number of different methods, including wireless methods (e.g., RF telemetry, ultrasound) or the two units could be hardwired together, e.g., with mating plugs or other releasably securable connections.

The Substrate and Reservoirs

The reservoir devices typically include a substrate having at least one reservoir, and more typically a plurality of reservoirs, containing reservoir contents to be selectively/controllably released or exposed. The reservoir devices in some embodiments further include one or more reservoir caps covering openings in the reservoirs. The reservoir caps may be designed and formed from a material which is selectively permeable to the molecules, which disintegrates/ruptures to release the molecules or, a combination thereof. Active release/exposure systems may further include control circuitry and a power source. U.S. Pat. Nos. 5,797,898, 6,123,861, 6,491,666, 6,527,762, 6,551,838, 6,875,208, 6,976,982, and 7,070,590, and U.S. Patent Application Publication No. 2004/0106953 to Yomtov et al., are incorporated herein by reference.

The substrate can be the structural body (e.g., part of a device) in which the reservoirs are formed, e.g., it contains the etched, machined, or molded reservoirs. In one embodiment, the device comprises a body portion, i.e., a substrate, that includes one or more reservoirs for containing reservoir contents sealed in a fluid tight or hermetic manner. As used herein, the term “hermetic” refers to a seal/containment effective to keep out helium water vapor, and other gases. As used herein, the term “fluid tight” refers to a seal/containment which is not gas hermetic, but which is effective to keep out dissolved materials in a liquid phase (by excluding the liquid), for example, an analyte to be measured by a sensor sealed in a reservoir. In a fluid tight, but non-hermetic device, water vapor could pass through a seal and re-condense, yielding liquid water inside a sealed reservoir or a sealed device; however, dissolved materials that could not form a gas at normal operating conditions such as in the body (e.g., salts, glucose) would not be able to pass through the fluid tight seal.

In one embodiment, the reservoirs are discrete, substantially non-deformable, and disposed in an array across one or more surfaces (or areas thereof) of the device body. As used herein, the term “reservoir” means a well, a cavity, a recess, or a hole (which may be a through-hole, i.e., an aperture) suitable for storing, containing, and releasing/exposing a precise quantity of a material, such as a drug formulation, or a secondary device, such as a sensor, or subcomponent. In one embodiment, the device includes a plurality of the reservoirs located in discrete positions across at least one surface of the body portion. In another embodiment, there is a single reservoir per each reservoir substrate portion; optionally two or more of these portions can be used together in a single device.

Reservoirs can be fabricated in a structural body portion using any suitable fabrication technique known in the art. Representative fabrication techniques include MEMS fabrication processes, microfabrication processes, or other micromachining processes, various drilling techniques (e.g., laser, mechanical, EDM, and ultrasonic drilling), and build-up or lamination techniques, such as LTCC (low temperature co-fired ceramics). The surface of the reservoir optionally can be treated or coated to alter one or more properties of the surface. Examples of such properties include hydrophilicity/hydrophobicity wetting properties (surface energies, contact angles, etc.), surface roughness, electrical charge, release characteristics, and the like. MEMS methods, micromolding, micromachining, and microfabrication techniques known in the art can be used to fabricate the substrate/reservoirs from a variety of materials. Other methods known in the art can also be used to form the reservoirs. See, for example, U.S. Pat. No. 6,123,861 and U.S. Pat. No. 6,808,522. Various polymer forming techniques known in the art also may be used, e.g., injection molding, thermocompression molding, extrusion, and the like.

In various embodiments, the body portion of the device comprises silicon, a metal, a ceramic, a glass, a polymer, or a combination thereof. Examples of suitable substrate materials include metals (e.g., titanium, tantalum, stainless steel, various other alloys such as cobalt-chrome, or platinum-iridium), ceramics (e.g., alumina, silicon nitride), semiconductors (e.g., silicon), glasses (e.g., Pyrex®, BPSG), and degradable and non-degradable polymers (e.g., silicones, expanded PTFE). Where only fluid tightness is required, the substrate may be formed of a polymeric material, rather than a metal or ceramic which would typically be required for gas hermeticity. It is noted, however, that polymeric devices may be made gas hermetic, if for example the polymeric material is a liquid crystal polymer of certain geometries or, alternatively or in addition, is provided with a metal or ceramic coating.

In one embodiment, each reservoir is formed of (i.e., defined in) hermetic materials (e.g., metals, silicon, glasses, ceramics) and is hermetically sealed by a reservoir cap. In one case, the substrate and reservoirs are formed from an SOI (silicon on insulator) material.

In one embodiment, the reservoirs are located at the tip portion of a lead. The tip portion may be made of a metal, silicon, a glass, a ceramic, or a combination thereof and may be shaped to have a curved, rounded, and/or elongated surface where the reservoirs are arrayed in and defined along the curved surface. Alternatively, the tip portion may be include a substantially planer substrate comprising an array of reservoirs, wherein the planar substrate is packaged in a catheter or in another elongated structure suitable for minimally invasive insertion into the body of a patient.

Desirably, the substrate material is biocompatible and suitable for long-term implantation into a patient. In a particular embodiment, the substrate is formed of one or more hermetic materials. The substrate, or portions thereof, may be coated, encapsulated, or otherwise contained in a hermetic biocompatible material (e.g., inert ceramics, titanium, and the like) before use. Non-hermetic materials may be completely coated with a layer of a hermetic material. For example, a polymeric substrate could have a thin metal coating. If the substrate material is not biocompatible, then it can be coated with, encapsulated, or otherwise contained in a biocompatible material, such as poly(ethylene glycol), polytetrafluoroethylene-like materials, diamond-like carbon, silicon carbide, inert ceramics, alumina, titanium, and the like, before use. In a particular embodiment, the substrate is hermetic—that is, impermeable at least during the time of use of the reservoir device—to the molecules to be delivered and to surrounding gases or fluids (e.g., water, blood, electrolytes or other solutions).

The surface of the device may be coated with one or more materials to provide an optimal interface between the implant device and the tissue at the site of implantation. Depending upon the particular site of implantation and function of the device, the coating material may promote or retard vascularization around the device, using techniques and materials known in the art. For example, one may want to generate vascularity around a device that is a subcutaneous or intra-peritoneal implant. In contrast, implants in contact with the heart or blood vessels typically will not need additional vascularity, but one will want to use non-thrombogenic materials or materials that resist platelet adhesion to reduce the incidents of thrombosis. See, e.g., U.S. Patent Application Publications No. 2005/0267440 A1 to Herman et al., and 2005/0112169 A1 to Brauker et al. Geometry also will be an important design consideration in a blood contacting device.

The substrate may be formed into a range of shapes or shaped surfaces. It can, for example, have a planar or curved surface, which for example could be shaped to conform to an attachment surface, such as the skin. In various embodiments, the substrate or the device is in the form of a planar chip, a circular or ovoid disk, an elongated tube, a sphere, or a wire. The substrate may be flexible or rigid. In one embodiment, the reservoirs are discrete, substantially non-deformable, and disposed in an array across one or more surfaces (or areas thereof) of an implantable medical device.

The substrate may consist of only one material, or may be a composite or multi-laminate material, that is, composed of several layers of the same or different substrate materials that are bonded together. Substrate portions can be, for example, silicon or another micromachined substrate or combination of micromachined substrates such as silicon and glass, e.g., as described in U.S. Patent Application Publication 2005/0149000 or U.S. Pat. No. 6,527,762. Representative examples of glasses include aluminosilicate glasses, borosilicate glasses (e.g., PYREX™), crystal glasses, etc. In another embodiment, the substrate comprises multiple silicon wafers bonded together. In yet another embodiment, the substrate comprises a low-temperature co-fired ceramic (LTCC) or other ceramic such as alumina. Ceramic substrates also could be formed using sintering and casting techniques known in the art. In one embodiment, the body portion is the support for a microchip device. In one example, this substrate is formed of silicon.

Total substrate thickness and reservoir volume can be increased by bonding or attaching wafers or layers of substrate materials together. The device thickness may affect the volume of each reservoir and/or may affect the maximum number of reservoirs that can be incorporated onto a substrate. The size and number of substrates and reservoirs can be selected to accommodate the quantity and volume of reservoir contents needed for a particular application, manufacturing limitations, and/or total device size limitations to be suitable for implantation into or onto a patient.

In a particular embodiment, a reservoir may have multiple openings to permit more rapid diffusion and/or flow of material into and out of the reservoir than with a single opening. For instance, if the secondary device is a chemical or biological sensor, and the device is part of an implantable medical device, then the larger area for mass transport provided by the multiple openings can facilitate more rapid contact of the sensor with an analyte, which would lead to better sensing functionality of the device (e.g., shorter response times, increased sensitivity, lower limits of detection, etc.). In another particular embodiment, a reservoir may have a large opening to provide a correspondingly large exposed surface area. The exposed surface area may be a particularly important variable for sensors, especially planar sensors. In such cases, the volume of reservoir optionally may be quite small. For example, a thinner substrate may be used to reduce the distance over which analyte molecules must travel from outside of the reservoir (e.g., in the body) to the surface of the sensor. This can help minimize any sensing lag caused by diffusion of the molecule of interest through the reservoir to the sensor surface. In one case, the substrate is approximately 100 microns and a reservoir is about 70 nanoliters.

The substrate can have one, two, three or more reservoirs. In various embodiments, tens, hundreds, or thousands of reservoirs are arrayed across the substrate. For instance, one embodiment of an implantable drug delivery device includes between 100 and 750 reservoirs, where each reservoir contains a single dose of a drug for release. In one sensing embodiment, the number of reservoirs in the device is determined by the operational life of the individual sensors. For sensing applications, the number of reservoirs also is highly dependent upon the size and volume of the individual sensors.

Each reservoir may have one opening or two or more openings which are sealed with a reservoir cap. The two or more openings may be opposed from one another on distal surfaces of the substrate or may be adjacent to one another on the same surface of the substrate. In certain alternative embodiments, the reservoirs have no reservoir caps, for example, in some cases where the reservoir contents include a release system for passive controlled release of one or more chemical molecules (e.g., drug molecules heterogeneously or homogeneously dispersed in a matrix material). In one case where a reservoir has two opposed openings, each of the openings may be sealed with a discrete reservoir cap, or alternatively, one of the openings may be sealed with a reservoir cap and the other opening may be sealed by a material that is intended to be permanent, i.e., it is designed not to be removed, degraded, permeabilized, or disintegrated during operation of the device.

In one embodiment, the reservoirs are microreservoirs. The “microreservoir” is a reservoir suitable for storing and releasing/exposing a microquantity of material, such as a drug formulation. In one embodiment, the microreservoir has a volume equal to or less than about 500 μL (e.g., less than about 250 μL, less than about 100 μL, less than about 50 μL, less than about 25 μL, less than about 10 μL, etc.) and greater than about 1 nL (e.g., greater than about 5 nL, greater than about 10 nL, greater than about 25 nL, greater than about 50 nL, greater than about 1 μL, etc.). The term “microquantity” refers to volumes from about 1 nL up to about 500 μL. In one embodiment, the microquantity is between about 1 nL and about 1 μL. In another embodiment, the microquantity is between about 10 nL and about 500 nL. In still another embodiment, the microquantity is between about 1 μL and about 500 μL. The shape and dimensions of the microreservoir can be selected to maximize or minimize contact area between the drug material (or sensor or other reservoir contents) and the surrounding surface of the microreservoir. Reservoir volumes less than 1 nL are envisioned and may be desirable with certain devices.

In one embodiment, the reservoir is formed in a 200-micron thick substrate and has dimensions of 1.5 mm by 0.83 mm, for a volume of about 200 nL, not counting the volume that would be taken up by the support structures, which may be about 20 to about 50 microns thick. Reservoirs also have been made in a 100-micron thick substrate (reservoir volume of about 70 nL) and in a 525-micron thick substrate (reservoir volume of about 600 nL).

In another embodiment, the reservoirs are macroreservoirs. The “macroreservoir” is a reservoir suitable for storing and releasing/exposing a quantity of material larger than a microquantity. In one embodiment, the macroreservoir has a volume greater than about 500 μL (e.g., greater than about 600 μL, greater than about 750 μL, greater than about 900 μL, greater than about 1 mL, etc.) and less than about 5 mL (e.g., less than about 4 mL, less than about 3 mL, less than about 2 mL, less than about 1 mL, etc.).

Unless explicitly indicated to be limited to either micro- or macro-scale volumes/quantities, the term “reservoir” is intended to encompass both.

The substrate may include reservoir cap support structures, with two or more reservoir caps covering the one or more opening(s) of a single reservoir, as described in U.S. Patent Application Publications No. 2006/0057737 and No. 2005/0143715 to Santini Jr., et al., which are incorporated herein by reference. Reservoir cap supports can comprise substrate material, structural material, or coating material, or combinations thereof. Reservoir cap supports comprising substrate material may be formed in the same step as the reservoirs. The MEMS methods, microfabrication, micromolding, and micromachining techniques mentioned above could be used to fabricate the substrate/reservoirs, as well as reservoir cap supports, from a variety of substrate materials. Reservoir cap supports comprising structural material also may be formed by deposition techniques onto the substrate and then MEMS methods, microfabrication, micromolding, and micromachining techniques. Reservoir cap supports formed from coating material may be formed using known coating processes and tape masking, shadow masking, selective laser removal techniques, photolithography, lift off, or other selective methods. See e.g., U.S. Patent Publications No. 2005/0143715 to Santini Jr., et al. and No. 2006/0105275 to Maloney et al., which are incorporated herein by reference.

A reservoir may have several reservoir cap supports in various configurations over its reservoir contents. For example, one reservoir cap support may span from one side of the reservoir to the opposite side; another reservoir cap support may cross the first reservoir cap support and span the two other sides of the reservoir. In such an example, four reservoir caps could be supported over the reservoir. In one embodiment for a sensor application (e.g., a glucose sensor), the reservoir (of a device, which may include only one reservoir or which may include two or more reservoirs) has two, three, or more reservoir openings and corresponding reservoir caps. The dimensions and geometry of the support structure can be varied depending upon the particular requirements of a specific application. For instance, the thickness, width, and cross-sectional shape (e.g., square, rectangular, triangular) of the support structures may be tailored for a particular drug release kinetics for a certain drug formulation or implantation site, or for certain transport properties for an analyte to be detected, etc.

Reservoir Contents

The reservoir contents are essentially any object or material that needs to be stored and isolated (e.g., protected from) the environment outside of the reservoir until a selected time point when its release or exposure is desired. In various embodiments, the reservoir contents include a quantity of drug or other chemical molecules, a secondary device, or a combination thereof.

Following reservoir activation (i.e., opening), the reservoir contents may be released from or may be retained (e.g., immobilized) in the reservoir, depending upon the particular reservoir contents and application. For example, a catalyst or sensor may not require release from the reservoir; rather their intended function, e.g., catalysis or sensing, will occur upon exposure of the reservoir contents to the environment outside of the reservoir after opening of the reservoir cap—and typically following ingress of one or more reactants or ingress of an analyte of interest. In an alternative case, the catalyst molecules or sensing component may be released from the opened reservoir, as would be typical when the reservoir contents comprise drug molecules, in order to exert a therapeutic effect on a patient. However, the drug molecules may be retained within the reservoirs for certain in vitro applications, such as drug screening activities like high-throughput screening or screening of molecule activity or stability when exposed to various chemicals, environmental conditions (e.g., pH), genetic materials, biowarfare agents, bacteria, viruses, or formulations.

Secondary Devices

As used herein, unless explicitly indicated otherwise, the term “secondary device” includes any device or a component thereof that can be located in a reservoir. Secondary devices are further described in U.S. Pat. No. 6,551,838 and in U.S. Patent Application Publication No. 2004/0248320, which are incorporated herein by reference.

In a particular embodiment, the secondary device is a sensor or sensing component thereof. As used herein, a “sensing component” includes a component utilized in measuring or analyzing the presence, absence, or change in a chemical or ionic species, energy, or one or more physical properties (e.g., pH, temperature, pressure, viscosity) at a site. Types of sensors include biosensors, chemical sensors, physical (e.g. mechanical) sensors, optical sensors, or any other sensor suitable for use in cardiovascular care. Examples of sensing components include components utilized in measuring or analyzing the presence, absence, or change in a drug, chemical, or ionic species, energy (or light), or one or more physical properties (e.g., pH, pressure, viscosity, flowrate) at a site. The secondary devices may be integral to the device or can be fabricated separately and added to the device. The device may be implantable in a patient (e.g., a human or other mammal). See, e.g., U.S. Patent Application Publications No. 2006/0076236 to Shah et al., No. 2006/0025748 to Ye et al., and No. 2005/0049472 to Manda et al., which are incorporated herein by reference.

As used herein, the term “biosensor” includes sensing devices that transduce the chemical potential of an analyte of interest into an electrical signal (e.g., by converting a mechanical or thermal energy into an electrical signal), as well as electrodes that measure electrical signals directly or indirectly. The biosensor may have a biological sensing/recognition element (e.g., an enzyme, an antibody) intimately connected to or integrated within a transducer. The biosensor may include an enzymatic and/or electrochemical sensor that effects sensing by oxidizing or reducing certain chemical species formed by the enzyme. The biosensor's aim typically is to produce a digital electronic signal that is proportional to the concentration of a specific chemical or set of chemicals. The electronic signal is the end result; any optical sensor might convert a chemical concentration to an optical signal (e.g., fluorescence) and a photodetector could produce the electrical signal. The biosensor also may measure some non-chemical, physiological parameter, such as with an indwelling pressure sensor. The biosensor may measure intrinsic electrical signals (EKG, ECG, EEG, or other neural signals), temperature, pH, viscosity, or mechanical loads on tissue structures at various in vivo locations (e.g., strain gauges).

In various embodiments, the biosensor may be one known in the art for use in measuring an analyte selected from dissolved and total amounts of carbon dioxide, carbon monoxide, ammonia, dioxygen, ethanol, ionized calcium, sodium ion, potassium ion, lithium ion, hydrogen ion, chloride ion, magnesium ion, ammonium ion, hydrogen peroxide, ascorbic acid, glucose, cholesterol, uric acid, esterified cholesterol, urea, BUN (blood urea nitrogen), creatinine, creatine, triglycerides, lactate, lactate dehydrogenase, creatine kinase, alkaline phosphatase, creatine kinase-MB, alanine transaminase, aspartate transaminase, bilirubin, amylase, lipase, vitamin K or other clotting factors, anti-clotting factors such as warfarin and heparin, troponin, CrCl microalbuminuria, hs-CRP, CD40L, BNP, NT-proBNP (as described in Morrow & Braunwald, “Future of Biomarkers in Acute Coronary Syndromes: Moving Toward a Multimarker Strategy,” Circulation 108:250-52 (2003)), carcinoembryonic antigen or other tumor antigens, illegal drugs, and various reproductive hormones such as those associated with ovulation or pregnancy.

The biosensor can be adapted to detect essentially any biomarker that can be prognostic or diagnostic for a medical condition, disease, etc. Preferred biomarkers are those that can be used to help direct a patient's therapy—e.g., closed loop therapy. Particularly preferred biomarkers are those that can be used for cardiovascular care.

Examples of configurations and methods for fabricating biosensor are described for example in U.S. Pat. No. 5,200,051 to Cozzette, et al. and U.S. Patent Application Publications No. 2006/0076236 to Shah et al., and No. 2006/0025748 to Ye et al., U.S. Pat. No. 6,978,178 to Sommer et al., U.S. Pat. No. 5,183,549 to Joseph et al., which are incorporated herein by reference. In one embodiment, the sensor may be fabricated as described in PCT WO 2005/075995 to Sphere Medical Ltd. and may include a biosensor that includes a molecularly imprinted polymer (MIP) as known in the art.

Exemplary sensors useful with the present reservoir devices for cardiac care applications may generally be divided into four categories: Those for detecting or measuring (1) electrolytes, (2) metabolites, (3) blood gases, or (4) macromolecules. Important electrolyte sensor applications include, but are not limited to, measuring blood pH, sodium ion concentration, potassium ion concentration, chloride ion concentration, calcium ion and total calcium concentrations. Sensing of these typically involves ISE or ISFET type sensors. Important metabolites to measure include, but are not limited to, glucose, lactate, creatinine, BUN, and bilirubin. Oxygen and carbon dioxide are important non-limiting examples of blood gases to quantify. Sensors for these utilize oxygen electrodes, potentiometric electrodes or FET. Important macromolecules to measure include, but are not limited to, alkaline phosphatase, aspartate aminotransferase, alanine aminotransferase, lactic dehydrogenase, gamma glutamyl transpeptidase, and heparin. Other examples of analytes which may useful to detect/measure with the biosensors of the present devices include, but are not limited to, ischemia modified albumin (IMA), myeloperoxidase, matrix metalloproteinase (MMP), and placental growth factor.

In one embodiment, the reservoir contents comprise at least one sensor indicative of a physiological condition in the patient. For example, the sensor could monitor the concentration of glucose, urea, lactate, calcium, or a hormone present in the blood, plasma, interstitial fluid, vitreous humor, or other bodily fluid of the patient. See, e.g. U.S. Patent Application Publication No. 2005/0096587 to Santini et al., which is hereby incorporated by reference. Information from the sensor could be used, for example, to actively control insulin release from the same device or from a separate insulin delivery device (e.g., a conventional insulin pump, either an externally worn version or an implanted version). Other embodiments could sense other analytes and deliver other types of drugs in a similar fashion.

The sensors of the present devices can be used to help monitor kidney unction, which may be particularly important to heart failure patients, as heart failure and kidney failure are often linked. Accordingly, the sensors may be designed to detect biomarkers for renal function, such as potassium, BUN, creatinine, and the like.

In one embodiment, the sensor is adapted to measure viscosity of blood or another physiological fluid in vivo. For example, U.S. Pat. No. 7,059,176 to Sparks describes a resonant tube viscosity sensing device. In one example, the sensor may be used to monitor blood coagulation by measuring the concentration of one or more anti-coagulants in the blood or by measuring blood viscosity or clotting time, or a combination thereof, using one or more sensors known in the art. See, Srivastava, Davenport, & Burns, “Nanoliter viscometer for analyzing blood plasma and other liquid samples,” Analytical Chemistry, 77(2):383-92 (2005). In one embodiment, blood viscosity could be measured to indicate a heart failure patient's fluid balance.

The sensor may be a pressure sensor, as described in U.S. Pat. Nos. 6,221,024, 6,237,398, and 6,706,005, and U.S. Patent Application Publication No. 2004/0073137, which are incorporated herein by reference. The sensor may include a cantilever-type sensor, such as those used for chemical detection, as described in U.S. Patent Application Publication No. 2005/0005676, which is incorporated herein by reference.

In another embodiment, the device is used in an ex vivo application to sense critical analytes or compounds. For example, sensors can be included in a dialysis cassette to monitor critical analytes or compounds during dialysis. In one case, the reservoir devices are integrated into a dialysis cassette and contain sensors. See, for example, U.S. Pat. No. 6,887,214 to Levin, which describes monitoring critical analytes or compounds such as metabolites, toxic materials, anti-coagulants, drugs, renal function indicators, phosphate, or biomarkers. A signal from the sensor may be transmitted (by any number of means, including hardwired or telemetry) to a separate molecule delivery device, which could also be located in a dialysis cassette.

In one embodiment it may necessary or desirable to inactivate the enzyme/biological recognition element of a spent sensor, particularly when that sensor may interfere with the operation of another sensor (i.e., in a different, nearby reservoir) that has been recently activated (i.e., reservoir opened and sensor exposed in vivo). Techniques and structures for destroying or deactivating reservoir based sensors are described, for example, in U.S. Patent Application Publication No. 2005/0096587 to Santini Jr. et al., which is incorporated herein by reference.

In another embodiment, the secondary device may be a MEMS device known in the art, such as a pressure sensor, an accelerometer, a gyroscope, a resonator, strain gauge, or the like.

Several options exist for receiving and analyzing data obtained with secondary devices located within the primary (multi-reservoir) device. The primary devices may be controlled by local microprocessors or remote control. Biosensor information may provide input to the controller to determine the time and type of activation automatically, with human intervention, or a combination thereof. For example, the operation of the device can be controlled by an on-board (i.e., within the package) microprocessor. The output signal from the device, after conditioning by suitable circuitry if needed, will be acquired by the microprocessor. After analysis and processing, the output signal can be stored in a writeable computer memory chip, and/or can be sent (e.g., wirelessly) to a remote location away from the reservoir device. Power can be supplied locally by a (standard or rechargeable) battery or remotely by wireless transmission. See, e.g., U.S. Patent Application Publication No. 2002/0072784. In one example, the electrical signal from a biosensor can be measured, e.g., by a mieroprocessor/controller, which then can transmit the information to a remote controller, another local controller, or both. For example, the system can be used to relay or record information on the patient's vital signs or the implant environment, such as drug concentration. Such information could be relayed to the patient's physician via the Internet, telephone, or radio signal, using devices and systems known in the art.

A device or system may have reservoir contents that include both drug molecules for release and a sensor/sensing component. For example, the sensor or sensing component can be located in a reservoir or can be attached to the device housing or located in another device. The sensor can operably communicate with the device, e.g., through a microprocessor, to control or modify the drug release variables, including dosage amount and frequency, time of release, effective rate of release, selection of drug or drug combination, and the like. The sensor or sensing component detects (or not) the species or property at the site of ex vivo release and further may relay a signal to the microprocessor used for controlling release from the device. Such a signal could provide feedback on and/or finely control the release of a drug. In another embodiment, the device includes one or more biosensors (which may be sealed in reservoirs until needed for use) that are capable of detecting and/or measuring signals within the body of a patient.

In one embodiment, the biosensor may be adapted for the detection of airborne analytes. Such embodiments could be useful, for example, in military and homeland defense applications, or other non-medical applications.

Drugs and Release-Controlling Materials

The reservoir contents may include essentially any substance for storage and controlled release. These substances may be stored in the reservoirs in essentially any form, such as a pure solid or liquid, a gel or hydrogel, a solution, an emulsion, a slurry, a suspension, or other mixtures. The substance of interest may be mixed with other materials to control the rate and/or time of release from an opened reservoir or enhance the stability, solubility, or complete release of the substance of interest. In various embodiments, the substance may be in the form of solid mixtures, including amorphous and crystalline mixed powders, monolithic solid mixtures, lyophilized powders, and solid interpenetrating networks. See, e.g., U.S. Patent Application Publications No. 2004/0247671 to Prescott et al. and No. 2004/0043042 to Johnson et al., which are incorporated herein by reference. In other embodiments, the substances are in a liquid-comprising form, such as solutions, emulsions, colloidal suspensions, slurries, or gel mixtures such as hydrogels.

In a particular embodiment, the reservoir contents include or consist of one or more drug formulations. The drug formulation is a composition that comprises a drug. As used herein, the term “drug” includes any therapeutic or prophylactic agent (e.g., an active pharmaceutical ingredient or API) as known in the art. In one particular embodiment, the drug is disposed in the reservoirs in a solid form, particularly for purposes of maintaining or extending the stability of the drug over a commercially and medically useful time, e.g., during storage in a drug delivery device until the drug needs to be administered. The solid drug formulation may be loaded into the reservoirs in a solid form or while in a liquid form, which is subsequently solidified/precipitated using processes such as drying or lyophilization. The solid drug matrix may be in pure form or in the form of solid particles of another material in which the drug is contained, suspended, or dispersed.

The drug can comprise small molecules, large (i.e., macro-) molecules, or a combination thereof. In various embodiments, the drug can be selected from amino acids, vaccines, antiviral agents, gene delivery vectors, interleukin inhibitors, immunomodulators, neurotropic factors, neuroprotective agents, antineoplastic agents, chemotherapeutic agents (e.g., paclitaxel, vincristine, ifosfamide, dacttinomycin, doxorubicin, cyclophosphamide, fluorouracil, carmustine, and the like), growth factors (e.g., fibroblast growth factors, platelet-derived growth factors, insulin-like growth factors, epidermal growth factors, transforming growth factors, cartilage-inducing factors, osteoid-inducing factors, osteogenin and other bone growth factors, and collagen growth factors), polysaccharides, anticoagulants and/or antiplatlet drugs (e.g., low molecular weight heparin, other heparins, aspirin, clopidogrel, lepirudin, fondaparinux, warfarins, dicumarol, pentasaccharides, etc.), antibodies, antibiotics (e.g., immunosuppressants), anti-microbials, analgesic agents (such as opioids and NSAIDS), anesthetics (e.g., ketoamine, bupivacaine and ropivacaine), anti-proliferatives, anti-inflammatories, angiogenic or anti-angiogenic molecules, and vitamins. In one embodiment, the large molecule drug is a protein or a peptide. Examples of suitable types of proteins include glycoproteins, enzymes (e.g., proteolytic enzymes), hormones or other analogs (e.g., luteinizing hormone-releasing hormone, steroids, corticosteroids, growth factors), antibodies (e.g., anti-VEGF antibodies, tumor necrosis factor inhibitors), bisphosphonates (e.g., pamidronate, clodronate, zoledronic acid, and ibandronic acid), tramadol, dexamethasone, cytokines (e.g., α-, β-, or γ-interferons), interleukins (e.g., IL-2, IL-10), diabetes/obesity-related therapeutics (e.g., insulin, exenatide, PYY, GLP-1 and its analogs). Any form of insulin, including short acting, long acting, etc. may be suitable for use with the present reservoir devices. The drug may be a gonadotropin-releasing (LHRE) hormone analog, such as leuprolide. The drug may be a parathyroid hormone, such as a human parathyroid hormone or its analogs, e.g., HPTH(1-84), HPTH(1-34), or hPTH(1-31). The drug may be selected from nucleosides, nucleotides, and analogs and conjugates thereof. The drug may be a peptide with natriuretic activity, such as atrial natriuretic peptide (ANP), B-type (or brain) natriuretic peptide (BNP), C-type natriuretic peptide (CNP), or dendroaspis natriuretic peptide (DNP). In still other embodiments, the drug is selected from diuretics, vasodilators, inotropic agents, anti-arrhythmic agents, Ca⁺ channel blocking agents, anti-adrenergics/ sympatholytics, and renin angiotensin system antagonists. The drug may be a vascular endothelial growth factor (VEGF) inhibitor, VEGF antibody, VEGF antibody fragment, or another anti-angiogenic agent. Examples include an aptamer, such as MACUGEN™ (Eyetech, pegaptanib sodium) or LUCENTIS™ (Genetech/Novartis, rhuFab VEGF, or ranibizumab). The drug may be a prostaglandin, a prostacyclin, or another drug effective in the treatment of peripheral vascular disease. The drug may be an angiogenic agent, such as VEGF. The drug may be an anti-inflammatory agent, such as dexamethasone. In one embodiment, the multi-reservoir device includes both angiogenic agents and anti-inflammatory agents. The drug may be selected from antiparasitic agents, antiviral agents, cytotoxins or cell proliferation inhibiting agents.

The drug may be a self-propagating agent, such as a gene therapy agent or vector. The drug may be in the form of cells, e.g., adult stem cells.

The drug may be in an encapsulated form. For example, the drug can be provided in microspheres or liposomes for controlled release. The drug may be provided in nanoparticle form.

In a particular embodiment, the substance for release includes may include an electrolyte (i.e., a salt for forming an aqueous solution of the salt), a metabolite, an anti-coagulant, erythropoietin, a red blood cell stimulating drug, or a molecule that may be depleted during dialysis. Such molecules are known in the art.

The reservoirs in one device can include a single drug or a combination of two or more different drugs, and may further include one or more pharmaceutically acceptable carriers. Two or more transport enhancers, angiogenic agents, anti-inflammatory agents, or combinations thereof, can be stored together and released from the same one or more reservoirs or they can each be stored in and released from different reservoirs.

The reservoirs in one device can include a single drug in two or more different formulations, for example to provide different dosing profiles over time. For example, different therapeutic or prophylactic agents, or different doses, can be delivered from a single device, either from the same surface region or from different surface regions. In one embodiment, the quantity of therapeutic or prophylactic agent provided for release from at least a first of the reservoirs is different from the quantity of the therapeutic or prophylactic agent provided for release from at least a second of the reservoirs. In another embodiment, the time of release of one of the therapeutic or prophylactic agents from at least a first of the reservoirs is different from the time of release of the therapeutic or prophylactic agent from at least a second of the reservoirs. In one embodiment, a first therapeutic or prophylactic agent is in at least one of the reservoirs and a second therapeutic or prophylactic agent is in at least one other of the reservoirs, the first therapeutic or prophylactic agent and the second therapeutic or prophylactic agent being different in kind or dose.

The drug or other substances for release can be dispersed in a matrix material to control the kinetics of release. The matrix material may be polymeric, non-polymeric, hydrophobic, hydrophilic, lipophilic, amphiphilic, and the like. The matrix may be bioresorbable or non-bioresorbable. For example, this matrix material can be part of a “release system,“as described in U.S. Pat. No. 5,797,898, which is incorporated herein by reference. The degradation, dissolution, or diffusion properties of the matrix material can provide a means for controlling, for example, the rate at which the chemical molecules is released from the reservoirs, the time at which release is initiated (e.g., following contact of the matrix material with a fluid outside of the reservoir), or both.

In one embodiment, release is initiated by degradation of the release system upon exposure to the carrier fluid. The chemical nature of the fluid, e.g., acid versus basic or polar versus non-polar, may cause the release system material, or matrix material thereof, to degrade or dissolve. The molecules of interest will be released into the carrier fluid flowing adjacent to the reservoir opening, as the matrix material is dissolved/degraded.

Particularly for drugs, the release system may include one or more pharmaceutical excipients. The release system may provide a temporally modulated release profile (e.g., pulsatile release) when time variation in plasma levels is desired or a more continuous or consistent release profile when a constant plasma level as needed to enhance a therapeutic effect, for example. Pulsatile release can be achieved from an individual reservoir, from a plurality of reservoirs, or a combination thereof. For example, where each reservoir provides only a single pulse, multiple pulses (i.e., pulsatile release) are achieved by temporally staggering the single pulse release from each of several reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of molecules through it over an extended period. In addition, continuous release can be approximated by releasing several pulses of molecules in rapid succession (“digital” release).

In certain embodiments, the chemical substance, e.g., drug, is formulated as a sustained or controlled release formulation. Sustained release materials known in the art are available for preparing compositions useful in the present devices. Exemplary materials include synthetic, biocompatible polymers known in the art. The polymer typically has a molecular weight greater than about 3000, more particularly greater than about 10,000, and less than about 10 million, more particularly less than about a million and even more particularly less than about 200,000. Non-limiting examples of polymers include poly-α-hydroxy acid esters, such as polylactic acid (PLLA or DLPLA), polyglycolic acid, polylactic-co-glycolic acid (PLGA), polylactic acid-co-caprolactone; poly (block-ethylene oxide-block-lactide-co-glycolide) polymers (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone; polyorthoesters; polysaccharides and polysaccharide derivatives such as polyhyaluronic acid, poly(glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins; polypeptides and proteins, such as polylysine, polyglutamic acid, albumin; polyanhydrides; polyhydroxy alkanoates such as polyhydroxy valerate, polyhydroxy butyrate, and the like.

In one embodiment, the drug formulation within a reservoir comprises layers of drug and layers of non-drug (i.e., matrix) material. After the active release mechanism has exposed the reservoir contents, the multiple layers provide multiple pulses of drug release due to intervening layers of non-drug. Such a strategy can be used to obtain complex release profiles. The technique could be used, for example, to deliver two different drugs that are incompatible with one another or otherwise should not be released at the same time. For instance, the layer structure could be non-drug/DrugA/non-drug/DrugB.

In another embodiment, the drug and matrix material can be provided in the reservoirs in a gradient form, where the concentration of the drug changes continuous with the depth in the reservoirs. For example, there may be a higher concentration of drug near one end (e.g., the end distal the opening of the reservoir) which decreases toward the other end. See, e.g., U.S. Patent Application Publication No. 2006/0147489, which is incorporated herein by reference.

The drug may be formulated with one or more excipients that facilitate transport through tissue capsules. Examples of such excipients include solvents such as dimethyl sulfoxide or collagen- or fibrin-degrading enzymes. See U.S. Patent Application Publication No. 2005/0267440 to Herman et al., which is incorporated herein by reference.

The drug may formulated with an excipient material that is useful for accelerating release, e.g., a water-swellable material that can aid in forcing the drug out of the reservoir, or otherwise provided in the reservoirs with components to effectuate more rapid release. See U.S. Patent Application Publication No. 2005/0055014 to Coppeta et al., which is incorporated herein by reference.

For in vitro applications, the chemical substances stored in the reservoirs can be any of a wide range of materials where the controlled release or exposure of a small amount (e.g., milligram to nanogram) of one or more types of molecules is required, for example, in the fields of analytic chemistry or medical diagnostics. The molecules may be effective as pH buffering agents, diagnostic reagents, and reagents in complex reactions such as the polymerase chain reaction or other nucleic acid amplification procedures. In various other embodiments, the molecules to be released are fragrances or scents, dyes or other coloring agents, sweeteners or other concentrated flavoring agents, or a variety of other compounds. In yet other embodiments, the reservoirs contain immobilized substances. Examples include any chemical species which can be involved in a reaction, including reagents, catalysts (e.g., enzymes, metals, and zeolites), proteins (e.g., antibodies), nucleic acids, polysaccharides, cells, and polymers, as well as organic or inorganic molecules that can function as a diagnostic agent, i.e., that are useful in diagnostic testing or imaging.

Release of the molecule from the reservoirs may be further controlled by the use of reservoir caps, including actively or passively reservoir disintegrated reservoir caps, or a combination of both actively and passively reservoir disintegrated reservoir caps, which are detailed below. For example, the reservoir cap may be removed actively to expose a passive release system, or a multi-reservoir device can include one or more passive release reservoirs and one or more active release reservoirs.

Reservoir Caps and Control Devices

As used herein, the term “reservoir cap” refers to a membrane, thin film, or other structure suitable for separating the contents of a reservoir from the environment outside of the reservoir, but which is intended to be removed, disintegrated, or permeabilized at a selected time to open the reservoir and expose its contents. Selectively removing or disintegrating the reservoir caps causes the contents of the reservoir to be exposed to the environment. As used herein, the term “disintegrate” includes degrading, dissolving, rupturing, fracturing or some other form of mechanical failure, as well as a loss of structural integrity due to a chemical reaction (e.g., electrochemical degradation) or phase change (e.g., melting) in response to a change in temperature, unless a specific one of these mechanisms is indicated. The disintegration of the reservoir cap may be by electrochemical activation as described in U.S. Pat. No. 5,797,898, by thermal activated from a separate heat source as described in U.S. Pat. No. 6,527,762, or by electrothermal ablation as described in U.S. Patent Application Publication No. 2004/0121486. (All of these patent publications are incorporated herein by reference.) As used herein, the term “environment” refers to the environment external to the reservoirs, including biological fluids and tissues at a site of implantation, air, carrier fluids, physiological fluids, and particulates present during storage or ex vivo use of a device as in transdermal or dialysis applications.

In a particular embodiment, a discrete reservoir cap completely covers one of the reservoir's openings. In another embodiment, a discrete reservoir cap covers two or more, but less than all, of the reservoir's openings. In still another embodiment, a reservoir has two or more openings, which are covered by two or more discrete reservoir caps.

In actively controlled devices, the reservoir cap includes any material that can be disintegrated or permeabilized in response to a suitable, applied stimulus (e.g., electric field or current, magnetic field, change in pH, or by thermal, chemical, electrochemical, or mechanical means). Examples of suitable reservoir cap materials include gold, titanium, platinumn tin, silver, copper, zinc, alloys, and eutectic materials such as gold-silicon and gold-tin eutectics.

In one embodiment, the reservoir caps are electrically conductive and non-porous. In a particular embodiment, the reservoir caps are in the form of a thin metal film. In another embodiment, the reservoir caps are made of multiple metal layers, such as a multi-layer/laminate structure of platinum/titanium/ platinum. For example, the top and bottom layers could be selected for adhesion layers on (typically only over a portion of) the reservoir caps to ensure that the caps adhere to/bonds with both the substrate area around the reservoir openings, reservoir cap supports, and a dielectric overlayer. In one case, the structure is titanium/platinum/titanium/platinum/titanium, where the top and bottom layers serve as adhesion layers, and the platinum layers provide extra stability/ biostability and protection to the main, central titanium layer. The thickness of these layers could be, for example, about 300 nm for the central titanium layer, about 40 nm for each of the platinum layers, and between about 10 and about 15 nm for the adhesion titanium layers. All of these thicknesses can be modified for a particular application or to accommodate various changes in the device structure (e.g., number of reservoirs, size and number of reservoir openings, and the like).

In passive devices, the reservoir caps are formed from a material or mixture of materials that degrade, dissolve, or disintegrate over time, or that do not degrade dissolve, or disintegrate, but are permeable or become permeable to molecules or energy. Representative examples of reservoir cap materials include polymeric materials and various types of semi-permeable membranes, and non-polymeric materials such as porous forms of metals (e.g., trabecular metal, a porous tantalum), semiconductors, and ceramics. Passive semiconductor reservoir cap materials include nanoporous or microporous silicon membranes. The reservoir cap material may be a porous silicon, such as a nanoporous silicon membrane (e.g., NANOGATE™ by Imedd Inc.) or a nanostructured porous silicon (e.g., BIOSILICON™ by Psividia Ltd.). NANOGATE™ is used as a non-degradable drug diffusion membrane, whereas BIOSILICON™ is used as a degradable matrix to release drug. The reservoir caps may be non-porous and formed of a bioerodible or biodegradable material, known in the art, such as a synthetic polymer, e.g., a polyester (such as PLGA), a poly(anhydride), or a polycaprolactone.

In one passive embodiment, release is initiated by degradation of the reservoir upon exposure to the carrier fluid. The chemical nature of the fluid, e.g., acid versus basic or polar versus non-polar, may cause the reservoir cap material to degrade or dissolve. Once the cap material is completely dissolved, the molecules will be released into the carrier fluid flowing adjacent to the reservoir opening. The fluid may be a liquid that causes the disintegration of the release system or the cap material or both.

In particular embodiments, the device may include a control device that facilitate and control reservoir opening, e.g., for disintegrating or permeabilizing the reservoir caps at selected times. The control device, as used herein, may comprise the structural components and electronics (e.g., circuitry and power source) for powering and for controlling the time at which release or exposure of the reservoir contents is initiated.

The control device can take a variety of forms. In one embodiment, the reservoir cap comprises a metal film that is disintegrated by electrothermal ablation as described in U.S. Patent Application Publication No. 2004/0121486 A1, which is incorporated herein by reference, and the control means includes the hardware, electrical components, and software needed to control and deliver electric energy from a power source (e.g., battery, storage capacitor) to the selected reservoir caps for actuation, e.g., reservoir opening. For instance, the device can include a source of electric power for applying an electric current through an electrical input lead, an electrical output lead, and a reservoir cap connected therebetween in an amount effective to disintegrate the reservoir cap. Power can be supplied to the control means of the multi-cap reservoir system locally by a battery, capacitor, (bio)fuel cell, or remotely by wireless transmission, as described for example in U.S. Patent Application Publication No. 2002/0072784.

The device may include a component to convert mechanical or chemical energy from the body of the human or animal into power (i.e. energy) which can be used to activate release or exposure of the reservoir contents. For example, components comprising accelerometers and gyroscopes can be used to convert motion of a body into electrical energy. Similarly, an implanted transducer can convert heartbeats into useful energy, as currently done with some pacemaker designs. See, e.g., U.S. Pat. No. 5,713,954.

A capacitor can be charged locally by an on-board battery or remotely, for example by an electromagnetic signal or ultrasound. The device may use acoustic communication and/or powering means, such as described in U.S. Pat. No. 7,024,248 to Penner et al., which is incorporated herein by reference.

In one embodiment, the control device includes an input source, a microprocessor, a timer, a demultiplexer (or multiplexer). The timer and (de)multiplexer circuitry can be designed and incorporated directly onto the surface of the substrate during fabrication. In another embodiment, some of the components of the control device are provided as a separate component, which can be tethered or untethered to the reservoir portion of the device. For instance, the controller and/or power source may be physically remote from, but operably connected to and/or in communication with, the multi-cap reservoir device.

In one embodiment, the power and electronics of the device are located in a housing and the reservoirs are disposed at the distal end portion of a catheter or lead, with wires connecting the reservoirs (i.e., the reservoir caps covering, and sensors located in, the reservoirs) to the housing.

In one embodiment, the operation of the multi-cap reservoir system will be controlled by an on-board (e.g., within an implantable device) microprocessor. In another embodiment, a simple state machine is used, as it typically is simpler, smaller, and/or uses less power than a microprocessor.

In one embodiment utilizing electrothermal ablation, the reservoir cap is formed of a conductive material adapted to have an electrical current passed through it to electrothermally ablate it. The reservoir cap is operably (i.e., electrically) connected to an electrical input lead and to an electrical output lead, to facilitate flow of an electrical current through the reservoir cap. When an effective amount of an electrical current is applied through the leads and reservoir cap, the temperature of the reservoir cap is locally increased due to resistive heating, and the heat generated within the reservoir cap increases the temperature sufficiently to cause the reservoir cap to be electrothermally ablated and ruptured. In this embodiment, the reservoir cap is formed of an electrically conductive material and the control circuitry comprises an electrical input lead connected to the reservoir cap, an electrical output lead connected to the reservoir cap, wherein the reservoir cap is ruptured by application of an electrical current through the reservoir cap via the input lead and output lead. In various embodiments, (i) the reservoir cap and the input and output leads may be designed to provide upon the application of electrical current an increase in electrical current density in the reservoir cap relative to the current density in the input and output leads, (ii) the material forming the reservoir cap has a different electrical resistivity, thermal diffusivity, thermal conductivity, and/or a lower melting temperature than the material forming the input and output leads, or (iii) the reservoir cap and the input and output leads are designed to provide upon the application of electrical current an increase in electrical current density in the reservoir cap relative to the current density in the input and output leads, and the material forming the reservoir cap has a different electrical resistivity, thermal diffusivity, thermal conductivity, and/or a lower melting temperature than the material forming the input and output leads.

Preferably, the control circuitry further comprises a source of electric power for applying the electrical current. Representative examples of suitable reservoir cap materials include gold, copper, aluminum, silver, platinum, titanium, palladium, various alloys (e.g., Au—Si, Au—Ge, Pt—Ir, Ni—Ti, Pt—Si, SS 304, SS 316), and silicon doped with an impurity to modulate the conductivity/resistivity because one can use the impurity to increase or decrease the conductivity or resistivity of the silicon, as known in the art. In one embodiment, the reservoir cap is in the form of a thin metal film. In one embodiment, the reservoir cap is part of a multiple layer structure, for example, the reservoir cap can be made of multiple metal layers, such as a multi-layer/laminate structure of platinum/titanium/platinum.

In another embodiment, the reservoir opening is closed by a reservoir cap comprising a dielectric or ceramic film layer and the actuation means comprises (i) an electrically conductive layer on top of the dielectric or ceramic film layer, and (ii) power source and control circuitry for delivering an electric current through the electrically conductive layer in an amount effective to rupture the dielectric or ceramic film layer, wherein the rupture is due to thermal expansion-induced stress on the dielectric or ceramic film layer. The electrically conductive layer and the actuation means can be designed to thermally ablate the electrically conductive layer or the electrically conductive layer could remain, in whole or in part, after rupturing the dielectric or ceramic film layer, depending on the particular design for opening/actuation the release of drug from the reservoir. See, e.g., U.S. Pat. No. 5,366,454 to Currie et al. and U.S. Pat. No. 6,114,658 to Roth et al.

In one embodiment, release may be in response to electrochemical stimulation. The application of an electrical potential causes the reservoir cap material to dissolve, providing for the release of the molecules into the liquid carrier fluid flowing adjacent to the reservoir opening. In a particular embodiment, the electric current would be modulated, rather than maintained at a constant value. See, e.g., U.S. Pat. No. 6,773,329.

In one embodiment, disintegration of the reservoir cap involves rupturing the reservoirs cap by application of a mechanical force generated from within or applied from outside of the reservoir. In such embodiments, the reservoir cap may be formed of a thin film of a metal or other material. In use, the mechanically rupturable reservoir caps may be ruptured by the pressure created by a pressurized reservoir pump such as an elastic bladder or a syringe pump, for example. The rupturable material can be selected from essentially any suitable brittle or fracturable material, such as titanium, tungsten, silicon, glass, or the like. The rupturable material also could be another type of material, such as a rubber or an elastomeric material with one or more defects engineered into it, which would cause the reservoir cap to fail by tearing/rupture. See, e.g., U.S. Pat. No. 7,052,488, U.S. Pat. No. 6,056,734 to Jacobsen et al., and U.S. Patent Application Publication No. 2005/0055014 to Coppeta et al.

In one embodiment, the device includes a substrate having a two-dimensional array of reservoirs arranged therein, reservoir contents contained in the reservoirs, discrete anode reservoir caps covering each of the reservoirs, cathodes positioned on the substrate near the anodes, and a control device for actively controlling disintegration of the reservoir caps. The control device includes a power source and circuitry to control and deliver an electrical potential; the energy drives a reaction between selected anodes and cathodes. Upon application of a potential between the electrodes, electrons pass from the anode to the cathode through the external circuit causing the anode material (reservoir cap) to oxidize and dissolve into the surrounding fluids, exposing or releasing the reservoir contents. The microprocessor directs power to specific electrode pairs through a demultiplexer as directed by an EPROM, remote control, or biosensor. Examples of reservoir cap materials in this embodiment include gold, silver, copper, zinc, and aluminum.

Possible reservoir opening and release control methods are further described in U.S. Pat. Nos. 5,797,898, 6,527,762, and 6,491,666, 6,808,522, 6,730,072, 6,773,429, 6,123,861; U.S. Patent Application Publication Nos. 2004/0121486, 2002/0107470 A1, 2002/0072784 A1, 2002/0138067 A1, 2002/0151776 A1, 2002/0099359 A1, 2002/0187260 A1, 2003/0010808 A1, 2002/0099359 A1, 2004/0082937 A1, 2004/016914 A1, 2006/0105275 A1, 2006/0057737 A1, 2005/0055014 A1, and 2006/0171989; PCT WO 2004/022033 A2; and PCT WO 2004/026281, all of which are incorporated by reference herein.

The reservoir control device can provide intermittent or effectively continuous release of the drug formulation. The particular features of the control device depend on the mechanism of reservoir cap activation described herein. For example, the control device can include an input source, a microprocessor, a timer, a demultiplexer (or multiplexer), and a power source. The power source provides energy to activate the selected reservoir, e.g., to trigger release of the drug formulation from the particular reservoir desired for a given dose. For example, the operation of the reservoir opening system can be controlled by an on-board microprocessor. The microprocessor can be programmed to initiate the disintegration or permeabilization of the reservoir cap at a pre-selected time or in response to one or more of signals or measured parameters, including receipt of a signal from another device (for example by remote control or wireless methods) or detection of a particular condition using a sensor such as a biosensor. In another embodiment, a simple state machine is used, as it typically is simpler, smaller, and/or uses less power than a microprocessor. The device also can be activated or powered using wireless means, for example, as described in U.S. 2002/0072784 A1 to Sheppard et al., which is incorporated herein by reference.

In one embodiment, the control device includes a microprocessor, a timer, a demultiplexer (or multiplexer), and an input source (for example, a memory source, a signal receiver, or a biosensor), and a power source. The timer and demultiplexer circuitry can be designed and incorporated directly onto the surface of the substrate during electrode fabrication, or may be incorporated in a separate substrate/device body. The microprocessor translates the output from memory sources, signal receivers, or biosensors into an address for the direction of power through the demultiplexer to a specific reservoir on the device. Selection of a source of input to the microprocessor such as memory sources, signal receivers, or biosensors depends on the microchip device's particular application and whether device operation is preprogrammed, controlled by remote means, or controlled by feedback from its environment (i.e., biofeedback). For example, a microprocessor can be used in conjunction with a source of memory such as erasable programmable read only memory (EPROM), a timer, a demultiplexer, and a power source such as a battery or a biofuel cell. A programmed sequence of events including the time a reservoir is to be opened and the location or address of the reservoir is stored into the EPROM by the user. When the time for exposure or release has been reached as indicated by the timer, the microprocessor sends a signal corresponding to the address (location) of a particular reservoir to the demultiplexer. The demultiplexer routes an input, such as an electric potential or current, to the reservoir addressed by the microprocessor. In another embodiment, the electronics are included on the substrate/chip itself, for example, where the electronics are based on diode or transistor technology known in the art.

In one particular embodiment, the electronics are separable from the reservoir device, such that they are reusable with the multi-reservoir pump devices. The cost to use a multi-reservoir pump device system like this would be significantly less than a system where the electronics were not separable and only could be used once.

Device Packaging and Housing

Embodiments of the reservoir device may be packaged with the control electronics and power supply as described in U.S. Pat. No. 6,827,250 to Uhland et al., U.S. Patent Publication No. 2005/0050859 to Coppeta et al., and U.S. Patent Application Publication No. 2006/0115323 to Coppeta et al., which are incorporated herein by reference.

The reservoir device may be contained with a device housing for ease of handling and protection of the components. The device housing may be formed from a variety of materials, such as polymers, metals, ceramics, and combinations thereof. In particular embodiments, the housing is formed of biocompatible materials, such as stainless steel, titanium or other inert materials known in the art.

### Methods of Using the Devices

Implantation of the Biosensor Device

In one case, the sensor device would be implanted and secured in a cardiac care patient. The implantation and securement of the biosensor could be carried out in a manner almost identical to that used to implant and secure a conventional pacemaker for in vivo operation, using techniques known in the art. Generally, this would entail a surgical procedure wherein a pocket would be made in the subcutaneous space using blunt dissention (creating a pocket between the skin and the muscle fascia). The implant device then would be placed into this pocket and secured with a few sutures, for example, through suture loops located on the device housing. If the sensor device is one in which the sensors are located at the end of a lead, then that lead would be placed in a blood vessel or in a heart chamber using conventional techniques for placement of a pacemaker lead or a defibrillator lead. A similar procedure would be used for embodiments where the sensor leads are added to another implantable cardiac device (e.g., a pacemaker or a defibrillator).

In one particular embodiment, the reservoir-based sensors described herein could be integrated into or the present sensor devices otherwise combined with a mesh sleeve that wraps around the heart. Such mesh wraps, which may be made of a flexible, biocompatible polyester mesh, are known in the art for the treatment of heart failure. For example, the mesh could be used to secure the tip portion of a lead against the outside of the heart. This may be useful where it is desired to measure a property on the external surface of the heart. One may assess, for example, ventricular function since the sensor would stay in relatively the same position relative to the heart muscle.

Use of Information from the Implanted Sensor

Information output from the implanted sensor can be used in essentially any way helpful to the patient, to the physician, or to a device useful in providing cardiac care (i.e., diagnosis or treatment or management of a cardiac disease, disorder, chronic condition, or failure). The sensor output desirably will be used to optimize delivery of medications to the patient. Various means of drug delivery, based on sensor output, are envisioned.

Transdermal Drug Delivery Coupled with Biosensor Device

In one embodiment, the present implantable biosensor devices are part of a transdermal pump drug delivery system. For instance, information from the implanted biosensor can be used to control the rate of drug delivered by the transdermal pump device.

The molecules contained in the reservoirs may be directly or indirectly pumped out of the multi-reservoir pump device using a variety of pumps, depending on the particular application. The pump can be essentially any pumping apparatus that causes a carrier fluid to flow through and out of the multi-reservoir pump device. The pump also could be one enabling an in-and-out flow, as with a membrane actuator or a synthetic jet type application, as described in U.S. Pat. No. 6,056,204. Pump apparatus suitable for use in these devices include elastic bladders, syringe pumps, membrane/diaphragm pumps, piston pumps with gas generating means, or peristaltic pumps containing a carrier fluid.

In one embodiment, the pump drives the carrier fluid across one or more surfaces of the substrate and reservoir caps or reservoir openings. For instance, a carrier fluid may be pumped so that it flows into a flow channel adjacent to a reservoir cap which is opened to release or expose the reservoir contents into the carrier fluid. In another embodiment, the pump provides backpressure on a flexible membrane covering an opening of the reservoir opposite a reservoir cap which may be disintegrated or made permeable to empty the molecules from the reservoirs. In yet another embodiment, the pump provides a carrier fluid through the reservoir which provides both backpressure to empty the molecules from the reservoirs and also a diluent in which the molecules may be dissolved.

The pump may be a peristaltic micropump. In one case, the pump may be driven by piezoelectric diaphragm actuators and may include back-pressure independent volumetric dosing with a pressure sensor for monitoring the dosing process and detecting catheter occlusions, as described in Geipel, et al., “Design of an Implantable Active Microport System for Patient Specific Drug Release” Proc. 24^{th} IASTED Int'l Multi-Conference Biomedical Engineering (February 2006, Innsbruck, Austria). See also U.S. Pat. No. 7,066,029, which describes methods and devices for monitoring fluid flow in vivo.

In a particular embodiment, the pump can be provided within a device housing also containing the reservoir device. See, e.g., U.S. Pat. No. 5,709,534 to O'Leary and U.S. Pat. No. 5,056,992 to Simons, which are incorporated herein by reference. In some embodiments, a pump can produce sufficient turbulence to mix the drug molecules from the reservoir and the carrier fluid sufficient to form a solution or ordered mixture. Sufficient turbulence also may be created by incorporating baffles within the flow channel and/or by adding a static or dynamic mixer/agitator.

The carrier fluid can be essentially of any composition in a fluid form suitable for being pumped in the devices described herein. As used herein, the term “fluid” includes liquids, gases, supercritical fluid, solutions, suspensions, gels, and pastes. In particular embodiments, the fluid is a non-gas, i.e., primarily includes one or more liquids, depending upon the particular device design and application.

Representative examples of suitable carrier fluids for medical applications include natural biological fluids and other physiologically acceptably fluids such as water, saline solution, sugar solution, blood plasma, and whole blood, as well as oxygen, air, nitrogen, and inhalation propellants. The choice of carrier fluid depends on the particular medical application, for example, transdermal drug delivery or sensing applications, dialysis applications, and the like.

In non-medical applications, the carrier fluid also can be selected from a wide range of fluids. Representative examples of suitable carrier fluids for use in fragrance release systems include water, organic solvents (such as ethanol or isopropyl alcohol), aqueous solutions, and mixtures of any of these. Representative examples of suitable carrier fluids for use in beverage additive systems include beverages or beverage bases of any type, such as water (both carbonated and non-carbonated), sugar solutions, and solutions of artificial sweeteners. In in vitro analytical or diagnostic applications, the carrier fluid may be essentially any chemical fluid. Examples include environmental samples of air or water, industrial or laboratory process sampling analysis, fluid samples to be screened in quality control assessments for food, beverage, and drug discovery, and combinatorial screening fluids.

The carrier fluid may be contained within the pump or may be stored in and supplied from a separate source. For example, in some embodiments, the pump may include an elastic bladder or a syringe and the carrier fluid may be contained within the elastic bladder or syringe. In one case, the pump may provide backpressure to empty the reservoir contents into a carrier fluid flowing across the reservoir openings from a carrier fluid source.

For embodiments in which the multi-reservoir pump device is intended for use in transdermal drug delivery or sensing applications, the device may be suitably (removably) secured to the site for the intended duration of use. Such securement features may be essentially any structure or material known in the art for securing objects to the skin of a patient. For example, the securement element can include one or more biocompatible adhesives, straps, or elastic bands. In one embodiment, the securement element is provided along the periphery of a housing of the device. An adhesive securement element can be, or can be readily adapted from, those known in the art for securing transdermal patches, such as those currently used in commercially available transdermal patches. See, e.g., U.S. Pat. No. 6,632,906, which is incorporated herein by reference.

In transdermal device embodiments, the skin-contacting surface desirably is flexible and hypoallergenic. The housing may further include other components, such as materials and structures for controlled delivery of an anesthetic agent or permeation enhancer.

In one embodiment, the adhesive is provided on a thin permeable material, such as a porous polymer layer, or a woven or non-woven fabric layer, which is adjacent the reservoir caps or the transport means. In one embodiment, the adhesive layer is permeable to the one or more pharmaceutical agents. In one embodiment, the polymer layer comprises a hydrogen In a particular embodiment, the securement element comprises a pressure sensitive adhesive, as known in the art.

In embodiments where the medical device comprises a transdermal multi-reservoir pump device, the device includes one or more conventional hypodermic needles, one or more microneedles, and/or one or more other needle means for transdermally delivering the carrier fluid and molecules into a patient's skin. Examples of microneedles suitable for transdermal drug delivery and analyte sensing are described in U.S. Pat. No. 6,743,211, U.S. Pat. No. 6,661,707, U.S. Pat. No. 6,503,231, and U.S. Pat. No. 6,334,856, all to Prausnitz et al., and in U.S. Pat. No. 6,230,051 and U.S. Pat. No. 6,219,574, both to Cormier et al, all of which are incorporated herein by reference. In optional embodiments, any other means known in the art of transdermal delivery may be used to enhance drug delivery through the stratum corneum, for example, by diffusion, capillary action, electroosmosis, electrophoresis, convection, magnetic field, ultrasound, or a combination thereof. These means may be used with, or in place of, one or more needles or microneedles.

### Methods for Manufacture or Assembly

The multi-reservoir devices may be made, for example, using techniques known in the art, particularly the methods described in U.S. Pat. No. 6,123,861 to Santini et al., U.S. Pat. No. 6,808,522 to Richards et al., U.S. Patent Application Publication No. 2004/0121486 to Uhland et al., U.S. Patent Application Publication No. 2006/0057737 to Santini Jr. et al., U.S. Patent Application Publication No. 2005/0096587 to Santini Jr. et al., U.S. Patent Application Publication No. 2006/0105275 to Maloney et al., which are each incorporated herein by reference.

The fabrication methods may use microfabrication and microelectronic processing techniques; however, it is understood that fabrication of device reservoir structures is not limited to materials such as semiconductors or processes typically used in microelectronics manufacturing. For example, other materials, such as metals, ceramics, and polymers, can be used to make the devices. Similarly, other fabrication processes, such as plating, casting, or molding, can also be used to make them.

In one embodiment, reservoirs may be formed using a silicon-on-insulator (SOI) techniques, such as described in S. Renard, “Industrial MEMS on SOI,” J Micromech. Microeng. 10:245-249 (2000). SOI methods can be usefully adapted to form reservoirs having complex reservoir shapes. SOI wafers behave essentially as two substrate portions that have been bonded on an atomic or molecular-scale before any reservoirs have been etched into either portion. SOI substrates easily allow the reservoirs (or reservoir sections) on either side of the insulator layer to be etched independently, enabling the reservoirs on either side of the insulator layer to have different shapes. The reservoir (portions) on either side of the insulator layer then can be connected to form a single reservoir having a complex geometry by removing the insulator layer between the two reservoirs using methods such as reactive ion etching, laser, ultrasound, or wet chemical etching.

Sealing the biosensors in the reservoirs and packaging the device for implantation is important for enabling the device to be suitable for long term implantation and operation of the biosensor devices. In a particular embodiment, the biosensors are sealed using a compression cold welding technique, which advantageously can avoid the application of heat which may be detrimental to many types of reservoir contents, such as sensitive sensor chemistries or proteins or peptide drugs. In a particular embodiment, the device includes at least two substrate portions bonded together as described in U.S. Patent Application Publication No. 2006/0115323 to Coppeta et al. The substrate portions include at least one cavity (i.e., a reservoir), which may be defined in one or both substrate portions. The space in the sealed cavity may be evacuated or may contain an inert gas or gas mixture (e.g., nitrogen, helium). In one case, the device includes contains a MEMS device, which may be on a third substrate. In another case, at least one of the bonded substrates is formed of a glass and the cavity contains an optical sensor or chemical compound that can be optically interrogated.

The reservoirs of the implant device may be sealed under vacuum or reduced pressure conditions, and/or with an inert gas, to enhance the stability of the reservoir contents (e.g., improve/extend molecular stability by slowing or preventing chemical degradation, such as by oxidation) and/or to accelerate the release or exposure of reservoir contents when the reservoir cap is removed (e.g., promote the ingress of any fluids in contact with the reservoir cap at the time the reservoir cap is removed). This technique may be useful for shortening the response time of a sensor within a reservoir. Representative examples of suitable inert gases include nitrogen (N₂), helium (He), argon (Ar), and combinations thereof Methods and equipment needed to provide and maintain a reduced pressure and/or inert gas blanket environment during the reservoir filling and device assembly processes, are know in the art. This sealing of the reservoir can be done by a variety of techniques, including those described in U.S. Pat. No. 6,827,250, U.S. Patent Application Publication No. 2005/0050859, and U.S. Patent Application Publication No. 2006/0115323 to Coppeta et al., which are incorporated herein by reference.

Publications cited herein are incorporated by reference. Modifications and variations of the methods and devices described herein will be obvious to those skilled in the art from the foregoing detailed description. Such modifications and variations are intended to come within the scope of the appended claims.

We claim:

1. An implantable medical device comprising:
a housing which includes a power source and control electronics;
at least one lead extending from the housing and having one or more discrete reservoirs therein, each reservoir having at least one opening to an outer surface of the lead;
one or more sensors, which monitor or detects in vivo an analyte, biomarker, or physical parameter that is associated with cardiac health, located in the one or more reservoirs and in operable communication with said control electronics; and
at least one selectively disintegratable reservoir cap sealing each of said at least one opening of said one or more reservoirs,
wherein the at least one reservoir cap is operably connected to the power source and control electronics to selectively disintegrate the reservoir cap and expose the one or more sensors in vivo.

2. The device of claim 1, wherein the one or more sensors comprises a biosensor for the monitoring or detecting of an analyte or a biomarker selected from the group consisting of potassium ion, sodium ion, lithium ion, magnesium ion, ammonium ion, ionized calcium, lactate, oxygen, carbon dioxide, creatinine, urea, BUN, bilirabin, alkaline phosphatase, aspartate aminotransferase, alanine aminotransferase, lactic dehydrogenase, gamma glutamyl transpeptidase, heparin, warfarin, ischemia modified albumin (IMA), myeloperoxidase, matrix metalloproteinase (MMP), pH, and placental growth factor.

3. The device of claim 1, wherein the one or more sensors or sensing components measures an ECG, an EKG, or another intrinsic electrical signal.

4. The device of claim 1, wherein the one or more reservoirs are located on a tip portion of the at least one lead.

5. The device of claim 1, wherein the at least one lead further comprises a secondary sensor located on an external surface of the at least one lead and not in the one or more reservoirs.

6. The device of claim 1, wherein the at least one lead further comprises an electrode for cardiac pacing, defibrillation, or neurostimulation.

7. The device of claim 1, wherein the at least one lead further comprises one or more secondary reservoirs containing at least one drug for controlled release in vivo.

8. The device of claim 7, wherein release of the at least one drug from the one or more secondary reservoirs is passively controlled.

9. The device of claim 7, wherein release of the at least one drug from the one or more secondary reservoirs is actively controlled.

10. The device of claim 1, further comprising at least one secondary lead without a sensor.

11. The device of claim 10, wherein the at least one secondary lead further comprises an electrode for cardiac pacing, defibrillation, or neurostimulation.

12. The device of claim 10, wherein the at least one secondary lead further comprises a plurality of drug reservoirs for controlled release of drug in vivo.

13. The device of claim 1, further comprising a transmitter for communicating an electrical signal from the one or more sensors to a remote receiver.

14. The device of claim 13, wherein the remote receiver is operably coupled to a controller for controlling delivery of a drug.

15. The device of claim 13, wherein the remote receiver is operably coupled to a controller for controlling cardiac pacing, defibrillation, or neurostimulation.

16. The device of claim 1, wherein the control electronics comprise a microprocessor or state machine.

17. A method of monitoring a patient in need of cardiac care comprising:
implanting into a patient a medical device which comprises
a housing which includes a power source and control electronics,
at least one lead extending from the housing and having one or more discrete reservoirs therein, each reservoir having at least one opening to an outer surface of the lead,
one or more sensors, which monitor or detects in vivo an analyte, biomarker, or physical parameter that is associated with cardiac health, located in the one or more reservoirs and in operable communication with said control electronics, and
at least one reservoir cap sealing each of said at least one opening of said one or more reservoirs, wherein the at least one reservoir cap is operably connected to the power source and control electronics;
opening the at least one opening of the at least one reservoir in response to the control electronics and exposing the one or more sensors in vivo; and
using the one or more sensors to monitor or detect an analyte, biomarker, or physical parameter associated with the health of the patient's heart.

18. The method of claim 17, wherein the analyte or biomarker is selected from the group consisting of potassium ion, sodium ion, lithium ion, magnesium ion, ammonium ion, ionized calcium, lactate, oxygen, carbon dioxide, creatinine, urea, BUN, bilirubin, alkaline phosphatase, aspartate aminotransferase, alanine aminotransferase, lactic dehydrogenase, gamma glutamyl transpeptidase, heparin, warfarin, ischemia modified albumin (IMA), myeloperoxidase, matrix metalloproteinase (MMP), pH, and placental growth factor.

19. The method of claim 17, wherein the physical parameter comprises a fluid flowrate, pressure, or viscosity.

20. The method of claim 17, wherein the physical parameter comprises an ECG, EKG, or another intrinsic electrical signal.

21. The method of claim 17, wherein the medical device further comprises an electrode for cardiac pacing, defibrillation, or neurostimulation.

22. The method of claim 17, wherein the medical device further comprises a drug for controlled release in vivo.

23. The method of claim 17, wherein the medical device comprises a plurality of discrete microreservoirs and a plurality of corresponding discrete reservoir caps which comprise a metal film and are mechanically and electrically connected to a pair of electrical leads.

24. The method of claim 17, wherein the step of opening the at least one opening of the at least one reservoir by permeabilizing or disintegrating the at least one reservoir cap.
